(19) United States
(12) Patent Application Publication
(10) Pub. No.: US 2026/0272629 A1
(43) Pub. Date: Sep. 17, 2026

(54) PISTON PUMP DRIVE ASSEMBLY FOR CLEANING AND CARE TOOLS

(71) Applicant: SHANGHAI SHIFT ELECTRICS CO., LTD., Shanghai (CN)

(72) Inventors: Xiaoguo DAI, Shanghai (CN); Zhenwu XU, Shanghai (CN)

(21) Appl. No.: 19/469,470

(22) PCT Filed: Mar. 27, 2023
(86) PCT No.: PCT/CN2023/083981

§ 371 Date: Sep. 26, 2025

(51) Int. Cl. A61C 17/02; A61M 11/00; F04B 17/00; F04B 53/14

(52) U.S. Cl. CPC A61C 17/0202; A61M 11/007; F04B 17/00; F04B 53/14

(57) ABSTRACT

The present disclosure discloses a piston pump drive assembly for a cleaning and care tool. The drive assembly includes a piston cylinder (8) and a piston (21). The piston is provided with a head sealing portion (211) capable of sealingly moving relative to an inner wall face of the piston cylinder, and a liquid inlet (23) and a liquid spray port (22) are provided in a head cavity (V2). The piston pump drive assembly further includes an electromagnetic drive device and at least one elastic element, where the electromagnetic drive device includes a coil and a magnet, the coil is fixed relative to the piston cylinder, and the magnet is located inside the cavity and fixedly attached to the piston.

### CROSS-REFERENCE TO RELATED APPLICATIONS

The present application is a U.S. National Stage Application under 35 U.S.C. § 371 of International Patent Application No. PCT/CN2023/083981, filed on Mar. 27, 2023, which is incorporated by reference in the present application in its entirety.

### FIELD

The present disclosure relates to the field of cleaning and care tools, and more particularly, to a piston pump drive assembly used in a cleaning and care tool.

### BACKGROUND ART

At present, piston pump drive mechanisms are used in personal care tools such as oral irrigators available on the market, to produce a jet of fluid for cleaning. A common piston pump drive mechanism uses a micro DC motor as a drive source for driving. Usually, the micro DC motor is connected to a gear set, a connecting rod is eccentrically installed on a driven gear of the gear set, and the connecting rod drives a piston to perform reciprocating linear movement in a piston cylinder.

In order to realize a connection between the connecting rod and the piston, the connecting rod needs to extend into the piston cylinder, and the connecting rod and the piston cylinder need to be sealed by a sealing member. Since the connecting rod is a moving component, dynamic sealing between the connecting rod and the piston cylinder is prone to fatigue yielding of the sealing member, resulting in a sealing failure.

In addition, the rotational speed of the micro motor decreases with the decrease of battery voltage, and there is an approximately linear relationship therebetween. A change in the rotational speed of the motor may change the number of water pulses per minute of an oral irrigator. Current medical experimental data show that the appropriate number of water pulses is very helpful for gum cleaning, and the micro motor may be affected by voltage fluctuations, which in turn causes fluctuations in the number of water pulses, thereby affecting the cleaning effect of the oral irrigator on gum cleaning. In addition, a mechanical fit between the gear set and the connecting rod may cause the oral irrigator to be noisy due to wear and tear, an excessive clearance, etc. Therefore, the existing piston pump drive assembly of the oral irrigator is still defective and needs to be improved.

### SUMMARY

To overcome the deficiencies in the prior art, the present disclosure provides a piston pump drive assembly, including: a piston cylinder, a cavity being formed inside the piston cylinder; and a piston, the piston being provided with a head sealing portion, the piston being arranged inside the cavity of the piston cylinder in a reciprocally movable manner in a longitudinal direction, the head sealing portion moves in a sealing manner relative to an inner wall face of the piston cylinder, the cavity including a variable-volume head cavity formed adjacent to the head sealing portion, and the head cavity being provided with a liquid inlet and a liquid spray port, where the piston pump drive assembly further includes an electromagnetic drive device and at least one elastic element, where the electromagnetic drive device includes at least one coil fixed relative to the piston cylinder, and further includes at least one magnet or at least one ferromagnetic element, the magnet or the at least one being located inside the cavity of the piston cylinder and fixedly attached to the piston, and the elastic element is arranged between the piston cylinder and the piston, to exert an elastic force on the piston during movement of the piston. Generally, a direction of the elastic force is substantially parallel to the longitudinal direction.

According to embodiment of the present disclosure, the electromagnetic drive device includes at least one magnet and at least one ferromagnetic element; where the at least one magnet further includes a first magnet and a second magnet, the first magnet and the second magnet being spaced apart and having like poles facing each other; and the at least one ferromagnetic element is arranged between the first magnet and the second magnet.

According to another embodiment of the present disclosure, the at least one coil includes a first coil, a second coil and a third coil, the first coil being located between the second coil and the third coil, an alternating current being applied to the first coil, a direct current being applied to the second coil and the third coil, and the direct current being applied and magnetic fields generated by the second coil and the third coil respectively are of the same polarity at opposite end faces of the second coil and the third coil.

According to an embodiment of the present disclosure, the piston includes a body portion and an end cap portion, the body portion having a blind hole forming the cavity, the blind hole being sealed on the side of the head cavity and opened on an opposite side, the magnet or the ferromagnetic element being inserted into the cavity from the other open side, and the end cap portion being hermetically fitted onto the blind hole to hermetically retain the magnet or the ferromagnetic element inside the cavity.

According to another embodiment of the present disclosure, the piston further includes a piston bolt for connecting the end cap portion to the body portion, the magnet or the ferromagnetic element has a central hole, and the magnet or the ferromagnetic element is sleeved on the piston bolt through the central hole.

According to another embodiment of the present disclosure, the piston further includes an elastic retainer, the elastic retainer being located inside the cavity of the piston and arranged between the end cap portion and the magnet or the ferromagnetic element.

According to another embodiment of the present disclosure, the piston further includes a tail sealing portion away from the head sealing portion, the piston cylinder includes a piston cylinder body and a piston cylinder end cap hermetically fastened to the piston cylinder body, the cavity further includes a tail cavity adjacent to the tail sealing portion and the piston cylinder end cap, and the elastic element includes a first coil spring and a second coil spring, each of the first coil spring and the second coil spring abutting against the piston and the piston cylinder, the first coil spring and the second coil spring applying opposite elastic forces to the piston, the first coil spring being arranged inside the tail cavity, one end of the first coil spring abutting against the piston cylinder end cap, the second coil spring is sleeved onto the piston close to the head cavity, one end of the second coil spring abutting against a stepped face on an inner wall of the piston cylinder, and the other end of the second coil spring abutting against a stepped face on an outer surface of the piston.

According to another embodiment of the present disclosure, the first coil spring and the second coil spring are always in a compressed state during the reciprocating movement of the piston inside the cavity of the piston cylinder.

According to another embodiment of the present disclosure, the piston further includes a tail sealing portion away from the head sealing portion, the tail sealing portion h moves in a sealing manner relative to the inner wall face of the piston cylinder, and the cavity of the piston cylinder further includes a tail cavity adjacent to the tail sealing portion. In one embodiment, the tail cavity is provided with at least one communication port.

According to another embodiment of the present disclosure, the at least one communication port includes a gas intake port and a gas exhaust port, where the gas intake port is attached to a gas intake check valve controlling unidirectional gas intake of the gas intake port, the gas exhaust port is in communication with a spray cavity downstream of the liquid spray port, and the gas exhaust port is attached to a gas exhaust check valve controlling unidirectional gas exhaust of the gas exhaust port.

According to another embodiment of the present disclosure, the at least one coil is wound on an outer wall face of the piston cylinder, the electromagnetic drive device includes only the magnet, i.e., does not include a ferromagnetic element, and a length of the coil in the longitudinal direction is greater than the sum of a length of the magnet in the longitudinal direction and a movement distance of a single stroke of the magnet inside the cavity in the longitudinal direction.

According to another embodiment of the present disclosure, the magnet is provided and a magnetic line of force of the magnet extends in a radial direction perpendicular to the longitudinal direction. In one embodiment, the magnet is provided and a magnetic line of force of the magnet is parallel to a magnetic line of force generated by the coil after an alternating current passes through the coil.

According to another embodiment of the present disclosure, an alternating current with a frequency f passes through the at least one coil; and the elastic element, the piston and the magnet or the ferromagnetic element constitute a resonator that resonates under an action of the current passing through the coil, and preferably, the resonator has an inherent frequency fg within a range of 75% to 125% of the frequency f of the current in the coil.

The piston of the piston pump drive assembly according to the present disclosure is internally provided with the magnet and the spring, the coil of the magnet surrounds the outer wall face of the piston cylinder, and the alternating current passes through the coil, thereby forming a resonant motion to drive the piston to efficiently perform reciprocating movement in the piston cylinder. Since the alternating current in the coil is fixed in frequency, and a movement frequency of the piston only follows the frequency of the current in the coil, the frequency of the reciprocating movement of the piston is fixed, and the unit time of water pulses excited by the piston to be ejected from a nozzle is fixed, thereby realizing a fixed number of the water pulses, and solving the problem in the prior art that the water pulses change with the change of battery voltage.

The reciprocating movement of the piston is driven by a non-contact electromagnetic force between the magnet and the coil, which solves the problem of an impact caused by an excessive clearance, etc. due to movement clearances, wear and tear, etc. between moving members during driving, and causes the piston to move more steadily. Since the impact is reduced, a high-frequency noise caused by the impact is also reduced.

In the embodiments of the present disclosure, dynamic sealing between the connecting rod and the sealing member is no longer adopted. The piston cylinder body and the piston cylinder end cap of the piston cylinder are connected by a fastener, and the inside of the piston cylinder can bear higher internal pressure, and in particular, the tail cavity can bear higher internal pressure, thereby realizing reliable sealing of the inside of the piston cylinder.

### BRIEF DESCRIPTION OF THE DRAWINGS

For a more complete understanding of the present disclosure, the following description of exemplary embodiments can be considered with reference to the drawings, in which:

FIG. 1 is a schematic diagram of a piston pump drive assembly and a nozzle for a cleaning and care tool according to an embodiment of the present disclosure;

FIG. 2 is a sectional view of the piston pump drive assembly and the nozzle shown in FIG. 1 according to an embodiment of the present disclosure;

FIG. 3 is a sectional view of a piston pump drive assembly according to a first embodiment of the present disclosure;

FIG. 4 is a sectional view of the piston pump drive assembly according to the first embodiment of the present disclosure, in which some parts are omitted;

FIG. 5 is a schematic diagram of a water path and a gas path of the piston pump drive assembly according to an embodiment of the present disclosure;

FIG. 6 is a schematic diagram of a water path and a gas path of the piston pump drive assembly according to another embodiment of the present disclosure;

FIG. 7 is a schematic diagram of a water path and a gas path of the piston pump drive assembly according to yet another embodiment of the present disclosure;

FIG. 8 is a sectional view of a piston pump drive assembly according to a second embodiment of the present disclosure; and

FIG. 9 is a sectional view of a piston pump drive assembly according to a third embodiment of the present disclosure.

### LIST OF REFERENCE SIGNS

1 Nozzle
2 Liquid inlet pipe
3 Gas intake pipe
4 Gas delivery pipe
5 Coil
6 Water inlet pipe connector
7 Gas delivery pipe connector
8, 8′, 8″ Piston cylinder
9 Piston cylinder end cap
10 End cap screw
11 Nozzle connector
12 Gas intake pipe connector
13 Gas intake pipe check valve
14 Water inlet pipe check valve
15 Gas delivery pipe check valve
16 Nozzle sealing member
17 Liquid spray port check valve
18 Second spring
19, 19′, 19″ Piston bolt
20 Magnet
20′-1, 20′-2 Magnet
20′, 20″ Ferromagnetic element
21, 21′, 21″ Piston
22 Liquid spray port
23 Liquid inlet
24 Gas delivery port
25 Piston cylinder tail gas inlet
26 Piston cylinder tail gas outlet
27 End cap portion
28 Elastic retainer
29 First spring
30 Magnet sealing member
31 Piston cylinder end cap sealing member
32 Water inlet pipe sealing member
33 Gas delivery pipe sealing member
34 Nozzle interface sealing member
35 Gas intake pipe sealing member
210 Tail sealing portion
211 Head sealing portion
V1 Tail cavity
V2 Head cavity
V3 Spray cavity
2′ Water inlet pipe
4′ Gas delivery pipe

### DETAILED DESCRIPTION OF EMBODIMENTS

The present disclosure will be further described below with reference to embodiments and drawings, and more details are set forth in the following description to facilitate a full understanding of the present disclosure. However, the present disclosure can obviously be implemented in many other ways than as described herein, and can make similar promotions and deductions based on practical applications without departing from the essence of the present disclosure. Therefore, the scope of protection of the present disclosure should not be limited to the content of the embodiments.

In the following descriptions, the structure of a cleaning and care tool is illustrated by taking an oral irrigator as an example, but it should be understood that the present disclosure may also be applicable to other personal cleaning and care tools that perform cleaning by using fluid pulses generated by piston pumps. It should be noted herein that the term “head/head end” in the following refers to an end or a side of a nozzle 1 close to the oral irrigator, and the term “tail/tail end” refers to an end or a side of the nozzle 1 away from the oral irrigator.

Each functional component of the oral irrigator is usually accommodated in a chamber enclosed by a shell of a handle portion. In one embodiment, the chamber of the handle portion accommodates a pipeline system for delivering a fluid, a piston pump drive assembly for providing a cleaning force to the fluid, a power supply (usually a rechargeable battery) for supplying power to the piston pump drive assembly, and a corresponding control device.

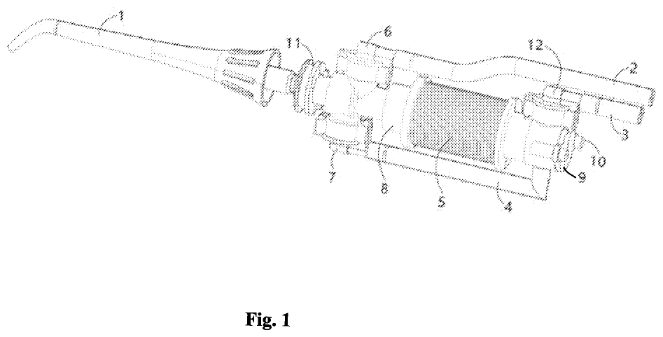

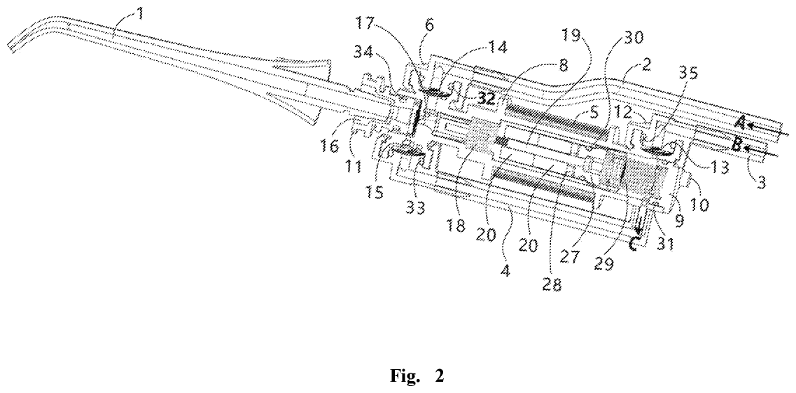

FIG. 1 shows a schematic diagram of a piston pump drive assembly and a nozzle 1 for a cleaning and care tool according to an embodiment of the present disclosure. FIG. 2 shows longitudinal sections of the piston pump drive assembly and the nozzle 1. As shown in FIGS. 1 and 2, the piston pump drive assembly mainly includes a piston cylinder 8, a piston 21, an electromagnetic drive device and an elastic element. The piston 21 is accommodated in a cavity inside the piston cylinder 8, and under the action of an electromagnetic force generated by the electromagnetic drive device and an elastic force of the elastic element, the piston 21 can perform reciprocating movement in a longitudinal direction of the piston 21 relative to the cavity of the piston cylinder 8, to realize a cyclic operation of fluid pumping.

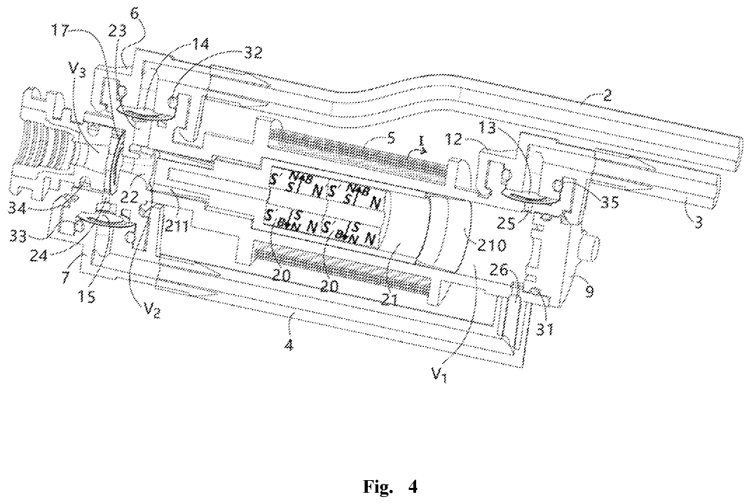

In one embodiment, the piston cylinder 8 is composed of a piston cylinder body and a piston cylinder end cap 9. The piston cylinder body and the piston cylinder end cap 9 are fixed together by means of one or more fasteners, such as end cap screws 10. The cavity inside the piston cylinder 8 is substantially cylindrical, as shown in FIG. 4, and the cavity is composed of cylindrical sections with different internal diameters. The shape of the piston 21 substantially conforms to the cavity of the piston cylinder 8, and the piston 21 also has sections with different external diameters, and the piston 21 may be fitted into the cavity of the piston cylinder 8.

In an embodiment, the piston 21 includes a head sealing portion 211 and a tail sealing portion 210. The head sealing portion 211 and the tail sealing portion 210 are provided with a circumferential head sealing face and a circumferential tail sealing face, respectively, and both of the two sealing faces may abut against a corresponding inner peripheral face of the cavity of the piston to form fluid sealing. As shown in FIG. 4, an external diameter of the head sealing portion 211 is less than an external diameter of the tail sealing portion 210.

The cavity formed inside the piston cylinder 8 is partitioned by the piston 21 to form a head cavity V2 and a tail cavity V1, with the head cavity V2 adjacent to the head sealing portion 211 and the tail cavity V1 adjacent to the tail sealing portion 210. The piston cylinder 8 is provided with a liquid spray port 22 and a liquid inlet 23 at the head cavity V2. The liquid spray port 22 is connected to the nozzle 1 by means of fluid communication through a nozzle connector 11, and a nozzle sealing member 16 may also be arranged between the nozzle connector 11 and the nozzle 1 to ensure that fluid sealing is formed therebetween. The liquid inlet 23 is provided for introducing a cleaning fluid (such as water or other functional cleaning fluids) into the piston cylinder 8.

During the use of the piston pump drive assembly, the volumes of the head cavity V2 and the tail cavity V1 are variable with the reciprocating movement of the piston 21. In one embodiment, as the piston 21 moves toward a tail of the piston cylinder 8, the head cavity V2 becomes larger in volume, the tail cavity V1 becomes smaller, and the fluid is pumped into the head cavity V2; and as the piston moves toward a head of the piston cylinder 8, the head cavity V2 becomes smaller in volume, the tail cavity V1 becomes larger in volume, and the fluid is ejected from the liquid spray port 22.

In particular, the movement of the piston 21 relative to the piston cylinder 8 is achieved by means of the electromagnetic drive device. The electromagnetic drive device mainly includes a coil 5 and a magnet 20. As shown in FIG. 2, the coil 5 is wound around an outer wall face of the piston cylinder 8, and preferably, the coil 5 is located between two flanges on the outer wall face. In another embodiment, the magnet 20 is fixed relative to the piston 21, and the magnet 20 is accommodated inside the cavity of the piston cylinder 8 together with the piston 21. After the electromagnetic drive device is energized and actuated, an electromagnetic force may be generated between the coil 5 and the magnet 20 to make the magnet 20 movable relative to the coil 5, thereby driving the piston 21 to move relative to the piston cylinder 8.

Further, in order to realize the reciprocating movement of the piston 21 relative to the piston cylinder 8, the elastic element is also arranged between the piston cylinder 8 and the piston 21. The elastic element is arranged between the piston cylinder 8 and the piston 21, to exert an elastic force on the piston 21 during movement of the piston 21. A direction of the elastic force is substantially parallel to the longitudinal direction in which the piston moves.

As shown in FIG. 2, in an embodiment of the present disclosure, the elastic element includes two coil springs 18 and 29, which are hereinafter referred to as a first spring 29 and a second spring 18 respectively.

The first spring 29 is arranged inside the tail cavity V1. The first spring 29 has one end abutting against the piston cylinder end cap 9, which is also referred to as a stationary end of the first spring 29, and has the other end abutting against a tail end of the piston 21, i.e., an end at which the tail sealing portion 210 is located. In one embodiment, the first spring 29 may abut against an end cap portion 27 of the piston 21.

At the second cavity V2 near the piston 21, the second spring 18 is sleeved on a section of the piston 21 having a smaller diameter. As shown in FIG. 2, one end of the second spring 18 abuts against a stepped face on an inner wall of the piston cylinder 8, and this end of the spring 18 is also referred to as a stationary end of the second spring 18, while the other end abuts against a stepped face on an outer surface of the piston 21, and this end of the spring 18 is a movable end. In this way, inside the first cavity V1, the first spring 29 arranged between the piston 21 and the piston cylinder 8 exerts an elastic force on the piston 21, the second spring 18 is arranged between the piston and the piston cylinder 8 closer to the head of the piston than the first spring 29 to exert an elastic force, and the elastic forces of the first spring 29 and the second spring 18 on the piston 21 are in opposite directions.

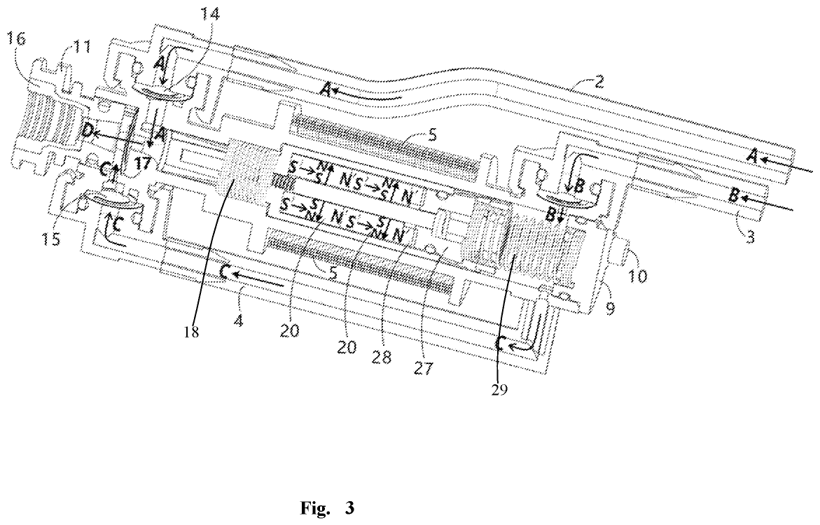

During the reciprocating movement of the piston 21 relative to the piston cylinder 8, the first spring 29 and the second spring 18 always abut against the piston 21 to be kept in a compressed state. For example, as shown in FIG. 3, the piston 21 moves to be closest to the nozzle 1, and the head cavity V2 is in its smallest state. At this moment, the second spring 18 is compressed to the maximum extent, and the first spring 29 is also in a compressed state but nearly in its relaxed state. In contrast, the tail cavity V1 is in its smallest state when the piston 21 moves to be furthest from the nozzle 1. At this moment, the first spring 29 is compressed to the maximum extent, and the second spring 18 is also in a compressed state. According to this embodiment, the first spring 29 and the second spring 18 are always in a compressed state, which can eliminate unnecessary noises caused by collisions of the piston cylinder 8 and the piston 21 with the springs 18, 29.

FIG. 3 shows a structure for securing the magnet 20 to the inside of the piston 21. In one embodiment, the piston 21 includes a body portion and an end cap portion 27 fitted at one end of the body portion. The body portion of the piston 21 has a blind hole forming the cavity, and the blind hole is sealed on the side of the head cavity V2 and opened at the tail cavity V1 at an opposite end. The end cap portion 27 is fitted to the open side of the blind hole. The magnet 20 is inserted into the cavity of the body portion from the open side, and then the end cap portion 27 is hermetically fitted onto the blind hole and the magnet 20 is hermetically retained inside the cavity. In one embodiment, in order to keep the cavity of the piston in a closed state to prevent any liquid from infiltrating into the cavity and hence causing corrosion to the magnet 20, a magnet sealing member 30 is arranged between the end cap portion 27 and an inner wall face of the cavity of the piston to realize a sealing connection of the end cap portion 27 relative to the piston 21.

The magnet 20 and the cavity of the blind hole are in the same shape and are both cylindrical. The magnet 20 may be one magnet or separable magnets 20.

In order to keep the magnet 20 fixed relative to the cavity of the blind hole, as shown in FIG. 2, the piston 21 further includes a piston bolt 19 for connecting the end cap portion 27 to the body portion. The piston bolt 19 extends through a bolt hole in the end cap portion 27 to a screw hole inside the cavity of the blind hole. The magnet 20 is provided with a central hole, and the magnet 20 is sleeved on the piston bolt 19 by means of the central hole, and the magnet 20 is retained between the end cap portion 27 and a bottom of the blind hole.

In addition, as shown in FIG. 2, the piston 21 further includes an elastic retainer 28. The elastic retainer 28 is arranged inside the cavity of the piston 21 and between the end cap portion 27 and the magnet 20, to facilitate retention of the magnet 20 and prevent the magnet from moving inside the cavity of the piston 21. The elastic retainer 28 is for example made of an elastomer material, for example, rubber.

As shown in FIG. 4, in an embodiment, a magnetic line of force inside the magnet 20 extends in a radial direction of the magnet, while a direction of a current in the coil 5, as shown in FIG. 4, is to flow into or out of paper, i.e., the current in the coil 5 flows around the magnet 20, and a direction of a magnetic field inside the coil 5, formed by the current in the coil 5, is parallel to the longitudinal direction of the magnet. A magnetic line of force outside the magnet 20 passes through the coil 5.

When an alternating current with a frequency f passes through the coil 5, the magnetic line of force outside the magnet 20 cuts the current-carrying coil 5 to produce a Lorentz force. Since the coil 5 is fixed to the piston cylinder 8, and the piston cylinder 8 is stationary relative to a shell of the oral irrigator, the coil 5 is also correspondingly constrained to remain stationary. The magnet 20 may be subjected to a reaction force of the Lorentz force, the magnet 20 may move relative to the piston cylinder 8, and the magnet 20 is driven by the force. In the state of FIG. 4, the magnet 20 is subjected to an electromagnetic force toward the left, and the magnet 20 moves to the left. Apparently, the force on the magnet 20 may also be reversed when the current in the coil 5 in FIG. 4 is reversed. That is, the magnet 20 is subjected to an electromagnetic force toward the right, and the magnet 20 moves to the right, thereby realizing the reciprocating movement.

In another embodiment, the magnet 20 inside the piston 21 may be provided and its magnetic line of force inside (N′, S′) is parallel to the magnetic line of force inside the coil, generated by the coil 5. In this case, the magnetic line of force of the magnet 20 does not cut the current carried by the coil. When the alternating current passes through the coil 5, an alternating magnetic field is generated inside the coil 5, and the magnetic line of force of the magnetic field inside the coil 5, formed by the current in the coil 5, is substantially parallel to the magnetic line of force generated by the magnet. For example, in the state of FIG. 4, the alternating current flowing through the coil 5 may generate a magnetic line of force from right to left inside the coil 5, while the magnetic line of force generated by the magnet 20 is in a left-to-right direction. The energized coil 5 generates an electromagnetic driving force toward the left on the magnet 20, to drive the magnet to move to the left and to further drive the piston to move to the left. When the current in the coil 5 in FIG. 4 is reversed, the current in the coil 5 generates a magnetic line of force from left to right inside the coil 5, while the magnetic line of force generated by the magnet 20 is in a left-to-right direction. The energized coil 5 generates an electromagnetic driving force toward the right on the magnet 20, to drive the magnet 20 to move to the right and to correspondingly drive the piston 21 to move to the right.

The first spring 29, the second spring 18, the magnet 20 and the piston that includes the piston bolt 19, the elastic retainer 28, the body portion and the end cap portion 27 constitute a resonator. At this moment, the resonator has an inherent frequency fg, and the magnitude of the inherent frequency fg of the resonator depends on the mass of the resonator and a resultant spring stiffness coefficient of the first spring 29 and the second spring 18. It is defined in engineering that the resonator is in a resonance state under the action of the driving force when fg lies between 75% and 125% of the frequency f of the driving force, and the resonator is in a resonant state under the action of the driving force when fg is equal to the frequency f of the driving force. The driving force is an acting force or reaction force between the magnet 20 and the coil 5, and the frequency of the driving force depends on the frequency of the current in the coil 5, i.e., the frequency of the driving force is equal to the frequency f of the current in the coil 5. In this embodiment, the inherent frequency fg of the resonator lies between 75% and 125% of the frequency f of the current in the coil, and the resonator resonates under the action of the driving force. In one embodiment, the inherent frequency fg of the resonator lies between 90% and 110% of the frequency f of the current in the coil, and the resonator resonates under the action of the driving force.

Since the frequency f of the current in the coil 5 is fixed, the movement frequency of the piston 21 is fixed, thereby achieving a fixed number of water pulses, which is more beneficial to the health of the gums. Moreover, in the resonance state, the mechanical efficiency of the piston pump drive assembly is greatly improved.

Advantageously, when the magnet 20 is arranged inside the coil 5 as shown in FIG. 2, the lengths of the magnet 20 and the coil 5 in the longitudinal direction should be chosen and the magnet 20 is always located inside the coil 5 in a cycle of motion. In other words, the magnet 20 does not exceed beyond the length of the coil 5 in the longitudinal direction. To this end, the length of the coil 5 in the longitudinal direction should be greater than the sum of a longitudinal length of the magnet 20 and a distance by which the magnet 20 moves in one direction relative to the coil 5.

The magnetic field intensity inside the coil 5 is proportional to the magnitude of the current in the coil 5. In the case where the magnet 20 is always located inside the coil 5, the magnitude of the electromagnetic force on the magnet 20 from the coil 5 is mainly affected by the magnitude of the current in the coil 5, but has little to do with the position of the magnet 20 inside the coil 5. In this way, the resonator is not disturbed by the position of the magnet when resonating under the electromagnetic force of the current in the coil 5.

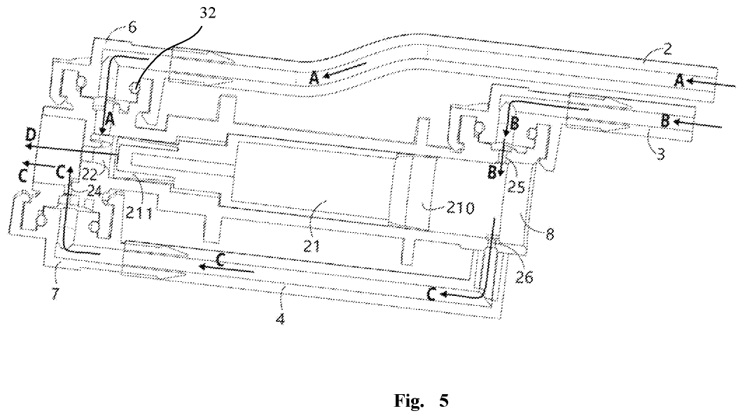

Next, a piping layout structure of the piston pump drive assembly is illustrated with reference to FIGS. 4 and 5. The cavity of the piston cylinder 8 includes a head cavity V2 and a tail cavity V1. A liquid inlet 23 of the head cavity V2 is connected to a water source, for example, a liquid reservoir, by means of a liquid inlet pipe 2. Between the liquid inlet 23 and the liquid inlet pipe 2, a water inlet pipe connector 6 is provided, and a water inlet pipe sealing member 32 is also provided. The sealing member 32 provides sealing between the water inlet pipe connector 6 and the piston cylinder body. As shown in FIG. 2, a water inlet pipe check valve 14 is arranged between the liquid inlet 23 and the water inlet pipe connector 6, and the water inlet pipe check valve 14 only allows a fluid outside the piston cylinder 8 to enter the head cavity V2, but does not allow the fluid to flow out of the head cavity V2. A liquid spray port check valve 17 is also arranged at the liquid spray port 22 of the head cavity V2. As shown in FIG. 4, the liquid spray port check valve 17 is arranged between the head cavity V2 and a spray cavity V3 downstream closer to the nozzle 1. The spray cavity V3 may be formed by a part of the piston cylinder 8, a part of the nozzle 1 or an end portion of a connector between the two, and the spray cavity V3 opens directly into the nozzle 1. The liquid spray port check valve 17 is provided to allow the fluid in the head cavity V2 to enter the spray cavity V3 downstream, but not to allow the fluid in the spray cavity V3 to return to the head cavity V2.

In addition, as shown in FIG. 4, a gas delivery port 24 is also provided at the spray cavity V3, and the gas delivery port 24 is connected to a gas outlet 26 of the tail cavity V1 by means of a gas delivery pipe 4. The gas delivery port 24 and the gas delivery pipe 4 are connected by means of a gas delivery pipe connector 7, and a gas delivery pipe sealing member 33 is also provided to prevent the fluid from overflowing. Similarly, a gas delivery pipe check valve 15 is arranged between the gas delivery port 24 and the connector 17. The gas delivery pipe check valve 15 is provided to allow only the fluid in the tail cavity V1 to enter the spray cavity V3, but to prevent the fluid from reversely flowing out of the cavity V3 along the gas delivery pipe 4.

In another embodiment, the tail cavity V1 is also provided with at least one port. In one embodiment, the tail cavity V1 is provided with two ports: a gas inlet 25 and a gas outlet 26. In one embodiment, both of the gas inlet 25 and the gas outlet 26 are arranged on a side wall of the piston cylinder body. However, it should be understood that in other embodiments, the gas inlet 25 and/or gas outlet 26 may also be arranged on the piston cylinder end cap 9.

As shown in FIG. 4, the gas outlet 26 of the tail cavity V1 is connected to the aforementioned gas delivery pipe 4. The gas inlet 25 of the tail cavity V1 is connected to a gas intake pipe 3 by means of a gas intake pipe connector 12, and a sealing member 35 is also arranged between the connector 12 and the piston cylinder 8 to ensure fluid sealing. The gas intake pipe 3 may lead to an external environment of the oral irrigator or to an open space inside the oral irrigator. At a position where the connector 12 is connected to the gas inlet 25, a gas intake pipe check valve 13 is arranged. The gas intake pipe check valve 13 is provided to allow only the fluid outside the piston cylinder 8 to enter the tail cavity V1, but not to allow the fluid in the tail cavity V1 to flow out reversely.

As shown in FIG. 5, when the piston pump drive assembly works, the pressure in the cavities V1 and V2 changes due to the reciprocating linear movement of the piston 21. Under the action of a pressure difference between atmospheric pressure and the pressure in the cavity V2, along a route A in FIG. 5, a liquid is pumped into the head cavity V2 from the liquid reservoir. Due to a pushing action of the piston 21, the liquid opens the liquid spray port check valve 17 to enter the spray cavity V3 and thus the nozzle 1, and is finally ejected through the nozzle 1 along a route D. In another embodiment, when the piston pump drive assembly works, the pressure in the tail cavity V1 changes due to the reciprocating linear movement of the piston. Along a route B including the gas delivery pipe 3, a gas is pumped into the cavity V1 from the outside of the piston cylinder 8 along the gas delivery pipe 3. Due to the pushing action of the piston, along a route C, the gas opens the gas delivery pipe check valve 15 by means of the gas delivery pipe 4 to enter the spray cavity V3 and the nozzle 1, and is finally ejected through the nozzle 1. In this way, the fluid sprayed by the nozzle 1 may be a mixture of gas and liquid, and the gas herein is usually an oxygen-containing gas. Injecting the oxygen-containing gas deep into a gingival sulcus can effectively remove anaerobic bacteria to help keep the gums healthy.

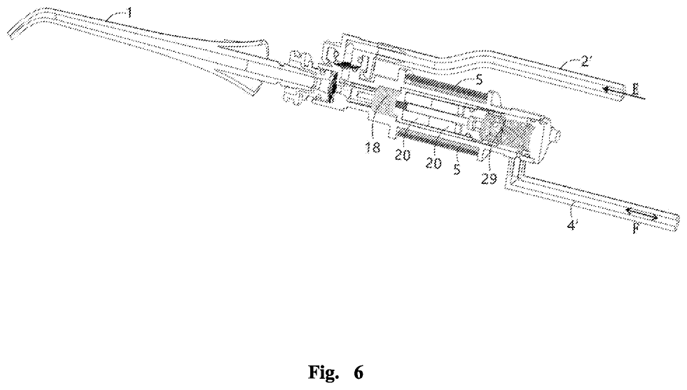

FIG. 6 shows a schematic diagram of a fluid pipeline of the piston pump drive assembly according to another embodiment. Different from the embodiment shown in FIG. 5, the tail cavity V1 is not in communication with the spray cavity V3, but is provided with a communication port. The communication port is in communication with atmosphere by means of a gas delivery pipe 4′. The communication port may be in direct communication with the outside of the shell of the oral irrigator, may also be in communication with a space inside the shell of the oral irrigator that is in communication with the external atmosphere, and is connected to the space inside the shell that is not affected by water. In this embodiment, it is unnecessary to provide a check valve or a flow limiting element on a communication path of the delivery pipe 4′.

In use of the piston pump drive assembly with the delivery pipe 4′ in communication with an atmospheric environment, as the piston 21 moves, the liquid in the liquid reservoir may enter the head cavity V2 along a water inlet pipe 2′ by following an arrow E. At the same time, as shown by a double-headed arrow F in FIG. 6, the gas in the tail cavity V1 may alternately enter and exit the tail cavity V1 along with the reciprocating movement of the piston 21. The delivery pipe 4′ in communication with the atmosphere plays a role of reducing the magnitude of the pressure change in the tail cavity V1. If the liquid that is supposed to be kept inside the head cavity V2 leaks into the tail cavity V1 due to a failure of sealing between the piston and the piston cavity, at least part of the liquid inside the tail cavity V1 may be sent out from the tail cavity V1 through the delivery pipe 4′.

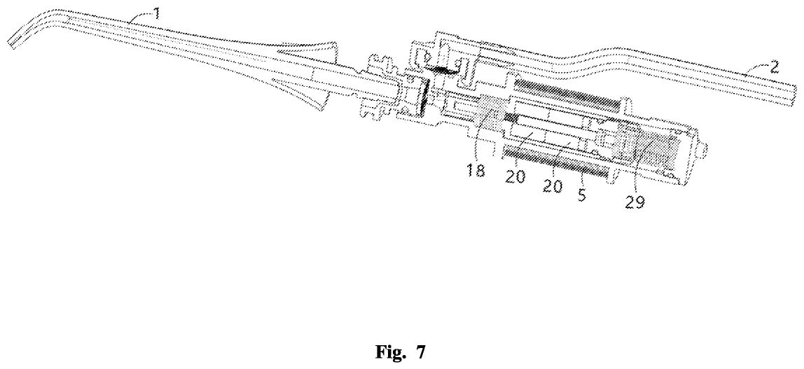

FIG. 7 shows a schematic diagram of a water path and a gas path of the piston pump drive assembly according to another embodiment. In the arrangement of the water path and the gas path shown in FIG. 7, the tail cavity V1 is not provided with a port in fluid communication with the outside of the cavity. In this case, the tail cavity V1 actually forms an isolated space. With this structure, the pressure in the tail cavity V1 may change dramatically with the reciprocating movement of the piston 21 relative to the piston cylinder 8. However, since the piston cylinder end cap 9 may be fixed to a tail end of the piston cylinder 8 by the end cap screw 10, and a sealing member 31 is arranged between the end cap 9 and the piston cylinder body, this structure can bear high cavity pressure and keep the tail cavity V1 hermetically isolated from the outside of the piston cylinder 8.

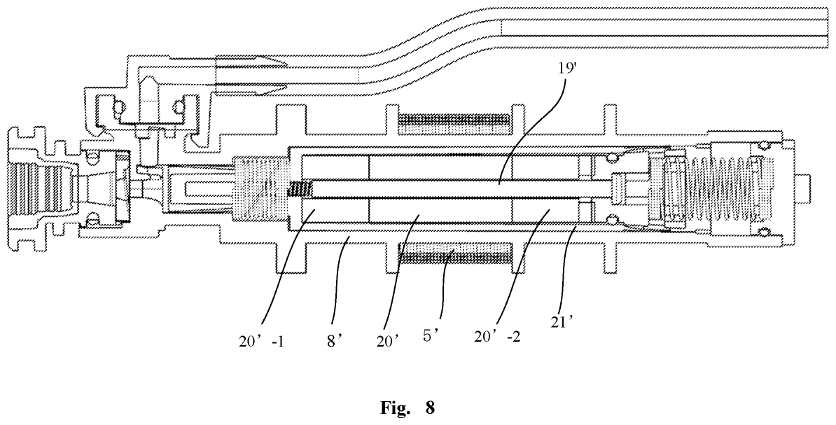

FIG. 8 shows a sectional view of a piston pump drive assembly according to a second embodiment of the present disclosure. As shown in FIG. 8, the arrangement of a piston cylinder 8′, a piston 21′ and an elastic element in the piston pump drive assembly is similar to that of the piston pump drive assembly in the first embodiment and thus will not be repeated herein.

In the embodiment shown in FIG. 8, an electromagnetic drive device in the piston drive assembly includes a coil 5′ and magnets 20′-1 and 20′-2. The coil 5′ is wound on an outer wall face of the piston cylinder 8′. In one embodiment, flanges that are spaced apart are integrally formed on the outer wall face of the piston cylinder 8′, and the coil 5′ is retained between the opposed flanges. The magnet 20′-1 and the magnet 20′-2 are arranged in a cavity portion formed inside the piston 21′. The two magnets 20′-1 and 20′-2 are arranged at a distance from each other and have like poles facing each other, with a ferromagnetic element 20′ arranged therebetween.

It should be noted that the magnet herein generally refers to a permanent magnet containing nickel or cobalt, while the ferromagnetic element refers to those materials that are magnetically conductive other than the permanent magnet, such as annealed iron and steel.

The manner in which the magnet 20′-1, the magnet 20′-2 and the ferromagnetic element 20′ are retained in the cavity inside the piston 21′ is similar to that in the first embodiment. Both of the magnet 20′-1 and the magnet 20′-2 have central holes, the central holes of the two magnets are aligned, the ferromagnetic element 20′ between the magnet 20′-1 and the magnet 20′-2 is provided with the same central hole, and a piston bolt 19′ passes through the central holes of the three and retains the three together by means of the end cap portion.

As can be seen from the longitudinal sectional structure shown in FIG. 8, in the longitudinal direction, the coil 5′ is arranged at a position approximately in the middle between the magnet 20′-1 and the magnet 20′-2. A length of the coil 5′ in the longitudinal direction is chosen and at least part of at least one of the magnets 20′-1 and 20′-2 is located outside a range occupied by the coil 5′ in the longitudinal direction. The magnet 20′-1 and the magnet 20′-2 are in the same shape and of the same size, and the magnets and the ferromagnetic element 20′ have the same diameter.

In use of the drive device in the embodiment, an alternating current with a frequency f passes through the coil 5′, and at this moment, a direction of the current in the coil 5′ is as shown in FIG. 8, i.e., flowing into paper or out of paper. Since the two magnets 20′-1 and 20′-2 have like poles facing each other, magnetic lines of force generated by the magnets 20′-1 and 20′-2 may turn to extend radially through the current-carrying coil 5′ after extending from their opposite end portions. The magnetic lines of force of the two magnets 20′-1 and 20′-2 cut the current-carrying coil 5′ to generate a Lorentz force. Since the coil 5′ is constrained to remain stationary, at this moment, the two magnets 20′-1 and 20′-2 are subjected to a reaction force of the Lorentz force and thus driven to perform relative reciprocating movement relative to the piston cylinder 8.

Compared with the first embodiment, the electromagnetic drive device having the two magnets 20′-1 and 20′-2 that are arranged opposite to each other is reduced in magnetic resistance, and the magnetic field intensity at the coil 5′ becomes higher. In addition, since the length of the coil 5′ is less than that of the ferromagnetic element, a magnetic line of force flowing out of or into the ferromagnetic element in a circumferential direction passes through the coil 5′. Since the magnetic lines of force are closed curves, in this embodiment, a magnetic line of force opposite to the magnetic line of force passing through the coil 5′ is away from the coil 5′, and the coil 5′ is relatively less affected by the force generated by the reversed magnetic line of force. Therefore, the electromagnetic drive device in the second embodiment can produce better driving efficiency.

Similar to the first embodiment, the two magnets 20′-1 and 20′-2, the ferromagnetic element, the piston including the piston bolt, the elastic retainer, the body portion and the end cap portion, and the springs at the two ends form a resonator, the resonator has an inherent frequency fg, fg should lie between 75% and 125% of the frequency f of the current in the coil 5′, and preferably, the inherent frequency fg of the resonator lies between 90% and 110% of the frequency f of the current in the coil, and the resonator resonates under the action of a driving force.

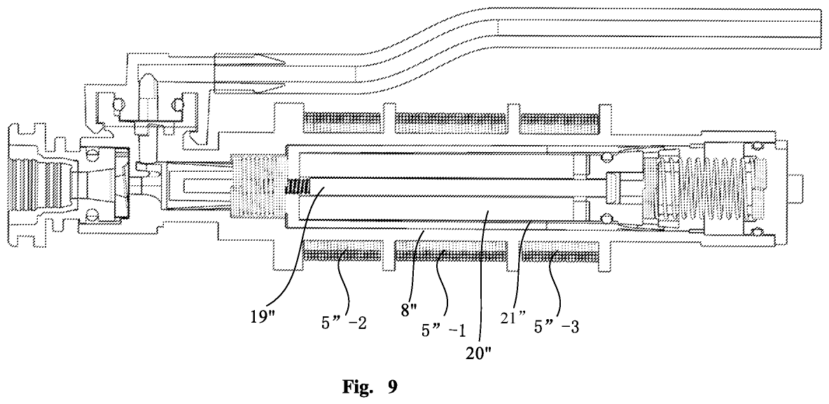

FIG. 9 shows a sectional view of a piston pump drive assembly according to a third embodiment of the present disclosure. As shown in FIG. 9, the arrangement of a piston cylinder 8″, a piston 21″ and an elastic element in the piston pump drive assembly is similar to that of the piston pump drive assembly in the first embodiment and the second embodiment and thus will not be repeated herein.

Particularly, in the piston pump drive assembly of the third embodiment, three coils are wound on an outer wall face of the piston cylinder 8″, namely a first coil 5″-1, a second coil 5″-2 and a third coil 5″-3, where the first coil 5″-1 is located between the second coil 5″-2 and the third coil 5″-3. An alternating current is applied to the first coil 5″-1, and a direct current is applied to the second coil 5″-2 and the third coil 5″-3. The second coil 5″-2 and the first coil 5″-1, as well as the third coil 5″-3 and the first coil 5″-1, may be separated by a flange integrally formed with the piston cylinder 8″. In one embodiment, longitudinal lengths of the second coil 5″-2 and the third coil 5″-3 are less than a length of the first coil 5″-1, while the second coil 5″-2 and the third line 5″-3 are of the same longitudinal length.

Instead of the magnet 20 in the first embodiment, in a third embodiment, a ferromagnetic element 20″ is attached relative to the piston 21″, and the ferromagnetic element 20″ may be a cylindrical integral member. In other alternative embodiments, the ferromagnetic element may also be formed by combining ferromagnetic elements 20″ connected together in series.

In one embodiment, a length of the ferromagnetic element 20″ in the longitudinal direction is greater than or equal to the sum of longitudinal lengths of the first, second and third coils.

As shown in FIG. 9, the ferromagnetic element 20″ has a central hole in the middle, the central hole is aligned with the cylindrical periphery of the ferromagnetic element 20″, and a piston bolt 19″ passes through the central hole, and the whole ferromagnetic element 20″ is fixedly accommodated in a cavity of the piston 21″.

During the use, an alternating current is applied to the first coil 5″-1, and a direct current is applied to the second coil 5″-2 and the third coil 5″-3. In this case, the second coil 5″-2 and the third coil 5″-3 are supplied with the direct current and two ends of the ferromagnetic element act like permanent magnets, the direct current is supplied and opposite end faces of the second coil 5″-2 and the third coil 5″-3 are of the same magnetic pole, and the resulting magnetic lines of force pass through the first coil 5″-1 located therebetween in a radial direction of the ferromagnetic element, and interact with the magnetic lines of force generated by the first coil 5″-1 through which the alternating current passes, to produce a force for reciprocating movement.

With the drive assembly of the third embodiment, the coil to which the direct current is applied is used instead of the magnet, and the cost of the whole assembly can be further reduced.

Similar to the first embodiment, the ferromagnetic element 20″, the piston 21″ including the piston bolt, the elastic retainer, the body portion and the end cap portion, and the springs at the two ends form a resonator, the resonator has an inherent frequency fg, fg should lie between 75% and 125% of the frequency f of the alternating current in the first coil 5″-1, and preferably, the inherent frequency fg of the resonator lies between 90% and 110% of the frequency f of the current in the coil, and the resonator resonates under the action of a driving force.

Other variations of the embodiments described above are also available.

For example, the elastic element of the piston pump drive assembly may be provided with only one spring, and may include only the spring 18 sleeving the piston. For example, the piston cylinder 8 may include only the head cavity, and the piston 21 includes only one sealing portion having a circumferential sealing face. The tail end of the piston cylinder 8 may be in an open state without forming a cavity.

In addition, the positions and structures of the check valves in all pipelines may also be changed according to connecting structures and extension directions of the pipelines.

Although the present disclosure is disclosed as above in terms of the embodiments, they are not intended to limit the present disclosure, and all possible changes and alterations can be made without departing from the spirit and scope of the present disclosure.

1. A piston pump drive assembly for a cleaning and care tool, comprising:
a piston cylinder, a cavity being formed inside said piston cylinder; and
a piston being provided with a head sealing portion, said piston being arranged inside said cavity of said piston cylinder in a reciprocally movable manner in a longitudinal direction, and said head sealing portion moves in a sealing manner relative to an inner wall face of said piston cylinder,
wherein said cavity comprises a variable-volume head cavity formed adjacent to said head sealing portion, and said head cavity is provided with a liquid inlet and a liquid spray port,
wherein said piston pump drive assembly further comprises an electromagnetic drive device and at least one elastic element,
wherein said electromagnetic drive device comprises:
at least one coil being fixed relative to said piston cylinder; and
at least one magnet or at least one ferromagnetic element being located inside said cavity and fixedly attached to said piston,
wherein said elastic element is arranged between said piston cylinder and said piston, so as to exert an elastic force on said piston during movement of said piston.

2. The piston pump drive assembly according to claim 1, wherein said electromagnetic drive device comprises at least one magnet and at least one ferromagnetic element;
said at least one magnet further comprises a first magnet and a second magnet being spaced apart and having like poles facing each other; and
said at least one ferromagnetic element is arranged between said first magnet and said second magnet.

3. The piston pump drive assembly according to claim 1, wherein said at least one coil comprises a first coil, a second coil and a third coil, said first coil being located between said second coil and said third coil;
an alternating current is applied to said first coil, and a direct current is applied to said second coil and said third coil; and
said direct current is applied such that magnetic fields generated by said second coil and said third coil respectively are of said same polarity at opposite end faces of said second coil and said third coil.

4. The piston pump drive assembly according to claim 1, wherein said piston comprises a body portion and an end cap portion, said body portion having a blind hole forming said cavity, said blind hole being sealed on a first side of said head cavity and opened on an opposite side, and said magnet or said ferromagnetic element being inserted into said cavity from a second open side; and
said end cap portion being hermetically fitted onto said blind hole to hermetically retain said magnet or said ferromagnetic element inside said cavity.

5. The piston pump drive assembly according to claim 4, wherein said piston further comprises a piston bolt for connecting said end cap portion to said body portion, said magnet or said ferromagnetic element has a central hole, and said magnet or said ferromagnetic element is sleeved on said piston bolt through said central hole.

6. The piston pump drive assembly according to claim 4, wherein said piston further comprises an elastic retainer being located inside said cavity of said piston and arranged between said end cap portion and said magnet or said ferromagnetic element.

7. The piston pump drive assembly according to claim 1, wherein said piston further comprises a tail sealing portion away from said head sealing portion, said piston cylinder comprises a piston cylinder body and a piston cylinder end cap hermetically fastened to said piston cylinder body, and said cavity further comprises a tail cavity (V1) adjacent to said tail sealing portion and said piston cylinder end cap, and
said elastic element comprises a first coil spring and a second coil spring, each of said first coil spring and said second coil spring abutting against said piston and said piston cylinder, and said first coil spring and said second coil spring applying opposite elastic forces to said piston,
wherein said first coil spring is arranged inside said tail cavity (V1), one end of said first coil spring abutting against said piston cylinder end cap; and
said second coil spring is sleeved on said piston close to said head cavity (V2), one end of said second coil spring abutting against a stepped face on an inner wall of said piston cylinder, and said other end of said second coil spring abutting against a stepped face on an outer surface of said piston.

8. The piston pump drive assembly according to claim 7, wherein said first coil spring and said second coil spring are always in a compressed state during said reciprocating movement of said piston inside said cavity of said piston cylinder.

9. The piston pump drive assembly according to claim 1, wherein said piston further comprises a tail sealing portion away from said head sealing portion, said tail sealing portion moves in a sealing manner relative to said inner wall face of said piston cylinder, said cavity of said piston cylinder further comprises a tail cavity (V1) adjacent to said tail sealing portion, and/or
said tail cavity is provided with at least one communication port.

10. The piston pump drive assembly according to claim 8, wherein said at least one communication port comprises a gas intake port and a gas exhaust port,
wherein said gas intake port is attached to a gas intake check valve controlling unidirectional gas intake of said gas intake port,
said gas exhaust port is in communication with a spray cavity (V3) downstream of said liquid spray port, and said gas exhaust port is attached to a gas exhaust check valve controlling unidirectional gas exhaust of said gas exhaust port.

11. The piston pump drive assembly according to claim 1, wherein said at least one coil is wound on an outer wall face of said piston cylinder, said electromagnetic drive device comprises only said at least one magnet, and a length of said coil in said longitudinal direction is greater than a sum of a length of said at least one magnet in said longitudinal direction and a movement distance of a single stroke of said magnet inside said cavity in said longitudinal direction.

12. The piston pump drive assembly according to claim 11, wherein
said magnet is provided such that a magnetic line of force of said magnet extends in a radial direction perpendicular to said longitudinal direction, or
said magnet is provided such that a magnetic line of force of said magnet is parallel to a magnetic line of force generated by said coil after an alternating current passes through said coil.

13. The piston pump drive assembly according to claim 1, wherein an alternating current with a frequency f passes through said at least one coil, and
said elastic element, said piston and said at least one magnet or said at least one ferromagnetic element constitute a resonator that resonates under an action of said current passing through said coil, and said resonator has an inherent frequency f_{g} within a range of 75% to 125% of said frequency f of said current in said coil.
